(19) United States
(12) Patent Application Publication
(10) Pub. No.: US 2013/0060282 A1
(43) Pub. Date: Mar. 7, 2013

(54) ORTHOPEDIC CHAIR FOR TREATMENT AND PREVENTION OF SPINAL DISEASES

(71) Applicant: Loan Kim Thi Pham, HCMC (VN)

(72) Inventor: Loan Kim Thi Pham, HCMC (VN)

(21) Appl. No.: 13/262,636

(22) PCT Filed: Sep. 3, 2011
(86) PCT No.: PCT/IB2011/053859

§ 371 Date: Mar. 23, 2012

(51) Int. Cl. A61F 5/01

(52) U.S. Cl. 606/242

(57) ABSTRACT

A chair comprises a seat and a seat back made of non-resilient materials.

The seat has a shape that matches the natural contours of an occupant's buttocks, thighs, and upper legs, wherein: The seat's concave backward portion connects to the concave lowest portion of the said seat back, creating a dedicated concave zone that contours the occupant's buttocks closely; the seat's concave backward portion has in its center a small hump that contours tightly an occupant's sacrum and coccyx if his buttocks are fitting inside the dedicated concave zone; the seat's forward portion has a wide W-shape that contours the occupant's upper legs: the two concave zones of the seat's W-shaped forward portion contours the back sides of the occupant's upper legs, the center zone of the seat's W-shaped forward portion is convex and snuggles the occupant's thighs; the seat is inclined backwardly in such a way that, when the occupant intentionally sits on the forward portion of the said seat, his buttocks, pushed by the gravity force of his body, will gradually glide backward and fit themselves into the said dedicated concave zone eventually; the left side and right side of the seat's W-shaped forward portion are convex so that they contour the outer sides of the occupant's upper legs.

The seat back contours the occupant's back, wherein: The seat back's lowest portion is concave to contour the occupant's buttocks; the middle portion that is convex to contour the lumbar vertebrae; the upper portion that is concave to contour the thoracic vertebrae; the left and right edges of the seat back are convex so that the occupant's back cannot bend left or right therefore must stay straight up; the seat back is height-adjustable to ensure that it well contours the occupant's spine; the seat back is positioned in a way that the occupant's back stays at an angle close to verticality, with his upper legs at an angle that is slightly greater than 90 degrees to the body's alignment.

### FIELD OF THE INVENTION

The present invention generally relates to a chair, and in particular, to an orthopedic chair that is used for prevention and treatment of spinal diseases including lumbar kyphosis, thoracic kyphosis, scoliosis, sacrum and coccyx misalignment, and herniated disc.

### BACKGROUND OF THE INVENTION

1. Spinal diseases arise from many different disorders, both postural and congenital. Several attempts have been made to treat these diseases, some of which were physical therapy or chiropractic care, bracing, surgery and various medications. Bracing is invented to support treatment of the spinal diseases. However, this invention is unattractive because it cannot be hidden, difficult to put on and uncomfortable to wear. Patients are required to wear their braces for at least 16 to 20 hours a day; therefore, while on, braces can interfere with the patients' daily activities and movements. Another downside of bracing is that it is unable to treat the spinal diseases. Patients have to turn to other treatment methods instead, such as surgery. Surgery may be able to treat the diseases and patients may get quick results; however, surgery is not always effective and not for everyone, especially not for those with osteoporosis or joint disease. In addition, surgery may lead to other complications such as infection, excessive bleeding, damages to nerves, and spinal cord. Drugs such as acetaminophen and NSAIDs can reduce pain; unfortunately, they can only relief pain temporarily. In addition to the drugs limited ability to treat spinal diseases, they cause liver and kidney damage, gastrointestinal bleeding, and ulcers. Physical therapy and chiropractic care seems to be safer options. Physical therapists train patients to change their postures. Chiropractors use spinal effective manipulation techniques, such as specific spinal manipulation or instrument-assisted manipulation, to treat the spinal diseases. However, in the treatment process, there is a risk of putting excessive forces on patients' spine that may lead to damaging it. On the other hand, if the chiropractors proceed with a safer approach by using less intensive forces, the treatment period is normally very lengthy. Plus, the treatment period is even prolonged given the fact that patients can only spend one to two hours a day with the chiropractors.

To treat the diseases safely and to shorten the treatment period, it becomes necessary to have a device that can replicate the safe forces applied by the chiropractors, and that is handy enough for patients to use it at home in order to increase the treatment time each day, thus to shorten the treatment period.

2. Given modern technologies, the numbers of hours people sit to do work surpass the number of hours people stand and walk around. Although a human body is designed for sitting, standing, and walking, it is not designed well enough to allow any human to engaged in any single activity for prolonged periods. Very frequently, people have bad sitting posture, such as sitting in backward or forward position, in which their spine is not provided with adaquate support.

In a backward sitting position, the spine is bent into a c-shaped curve, with the pelvis pushed forward. The weight of the chest and head is in front of the spine, increasing its tendency to bend. The ischial tuberosities, which takes most of the weight, are a long way in front of the spine, therefore the pelvis tends to rotate backwards. This tends to slide the legs along the seat cushion, making an occupant's posture unstable and creating a vicious circle as the pelvis rotates more. Also the skin under the sitter's thighs and buttocks is being dragged, which research shows makes it 50% less able to cope with the weight on it. As the pelvis rotates, the coccyx is moved underneath and the tip can be loaded painfully in bend on the seat cushion.

Also in backward sitting position, the buttock muscles move underneath and the constant pressure on them inhibits their blood supply, causing “numb bum”. If the sitter continues to practice this sitting posture, vertebral discs, due to constant pressure from the weight and the abnormal curve of the spine, will eventually degenerate resulting in severe back pain. In addition, the pressure eventually pinch the spinal cord and spinal nerve roots, which will not only cause pain but also weakness in the lower extremities. The change in spinal curvature will not only affect an individual's posture, it will also affect other parts of his or her body, especially the internal organs such as the lungs. When the spine is in kyphosis, the rib cage cannot expand to its maximum and the diaphragm cannot fully expand or contract. A decrease in respiratory efficiency results in a decrease in the functions of other parts of the body because human relies on oxygen to survive.

In a forward sitting position, the sacrum and the coccyx bend backwardly. Overtime, they bend backwardly permanently, a.k.a. misalignment, which leads to pinching nerves and herniated disc.

It is clearly now that a correct sitting posture is important. A correct sitting posture typically sees the back at an angle close to verticality, with the upper legs at an angle that is slightly greater than 90 degrees to the body's alignment. A correct sitting posture not only minimizes the risk of getting herniated disc, pinched nerve, “numb bum”, back pain and weakening of the lower extremities, it also improves respiratory efficiency. Many chair inventions have been produced with a single aim of correcting people's sitting posture.

A few previous inventions were on chairs that have a seat that inclines forward and therefore, have a knee rest to prevent the occupants from sliding off the seat. One of the disadvantages of these chairs is that occupants get pain in the knee due to constant pressures exerted on the knees.

Australian patent AU-B-73415/87 also described a chair for supporting correct posture. This chair has a seat comprising a backward portion made of materials that has low resistance to deformation and a forward portion made of materials with higher resistance to deformation; the two portions are divided by a crevice. With that, an occupant, to make himself comfortable, tends to keep his buttocks on the backward portion, resulting that he is sitting in a correct posture. However, due to its relatively higher resistance to resilient deformation, the forward portion can exert a pressure on the back of the upper legs that leads to impedance of the peripheral blood circulation in this area and therefore numbness may occur.

Australian Application No. 29072/99 described a “Soft Cell” chair. This chair seeks to address the mentioned disadvantage of the seat described in Australian patent AU-B-73415/87, by having a seat with the forward portion softer than the back portion. However, the biggest disadvantage of this chair is that it does not support correct posture.

In summary, it is necessary to develop a chair that is capable of treating spinal diseases effectively but comfortably and is suitable for daily use for supporting a correct sitting posture to prevent spinal diseases.

### SUMMARY OF THE INVENTION

The present invention provides an orthopedic chair that is capable of treating and preventing spinal diseases. The said orthopedic chair comprises a seat and a seat back. The said seat is mounted on a support that can include well-known height adjustment means; the support will not be described herein as it is not included in the invention.

The said seat has a shape that matches the natural contours of an occupant's buttocks, thighs, and upper legs. In which, the said seat has its concave backward portion connect to the concave lowest portion of the said seat back, creating a dedicated concave zone that contours the occupant's buttocks closely but comfortably. In the center of the seat's concave backward portion is a small hump that contours tightly the occupant's sacrum and coccyx if his buttocks are fitting inside the dedicated concave zone.

The forward portion of the said seat has a wide W-shape that contours the occupant's upper legs comfortably. In which, the two concave zones of the seat's W-shaped forward portion contours the back sides of the occupant's upper legs. The center zone of the seat's W-shaped forward portion is convex and has two functions. The first function is to snuggle the occupant's thighs comfortably. The second function is to encourage the occupant to fit his buttocks inside the dedicated concave zone, as sitting on the convex center zone of the W-shaped forward portion is not comfortable.

Furthermore, to ensure that the occupant always fit his buttocks inside the dedicated concave zone, the seat is designed to be inclined backwardly in such a way that, when the occupant intentionally sits on the seat's W-shaped forward portion, his buttocks, pushed by the gravity force of his body, will gradually glide backward and fit themselves into the said dedicated concave zone eventually.

The left side and right side of the seat's W-shaped forward portion are convex so that they contour the outer sides of the occupant's upper legs. Moreover, the convex left and right sides of the seat's forward portion keep the upper legs in a correct position, i.e. inside the two concave zones of the seat's W-shaped forward portion, since putting the legs on top of the two convex left and right sides is not comfortable.

The said seat back is designed in such a way that it contours the occupant's back tightly but not forcefully so that the occupant can sit in a correct posture comfortably. The said seat comprises a lowest portion, as already described above, that is concave to contour the occupant's buttocks, a middle portion that is convex to contour the lumbar vertebrae, and an upper portion that is concave to contour the thoracic vertebrae. The left and right edges of the seat back are convex so that the occupant's back cannot bend left or right therefore must stay straight up. The seat back is height-adjustable to ensure that it well contours the occupant's spine. Besides, the seat back is positioned in a way that the occupant's back stays at an angle close to verticality, with his upper legs at an angle that is slightly greater than 90 degrees to the body's alignment.

Here, a proper sitting posture is defined as a posture in which the occupant sits on the seat of the said chair in such a way that his buttocks fit inside the dedicated concave zone, his sacrum and coccyx are contoured tightly by the small hump in the center of the seat's concave backward portion, his two upper legs fit inside the two concave zones of the seat's W-shaped forward portion and is at an angle that is slightly greater than 90 degrees to the body's alignment, and his back stays at an angle close to verticality and is contoured tightly by the seat back. When an occupant is sitting in this proper sitting position, his pelvis is in neutral position, i.e. not rotating backward or forward, his spine is in its natural “S” shape and is not bent, and his sacrum and coccyx are not moved underneath.

The brief description above sets forth rather broadly the more important features of the present invention in order that the detailed description thereof that follows may be better understood, and in order that the present contributions to the art may be better appreciated. There are, of course, additional features of the invention that will be described hereinafter and which will be for the subject matter of the claims appended hereto. Before explaining the invention in detail, it is to be understood that the invention is not limited in its application to the details of construction and to the arrangements of the components set forth in the following description or illustrated in the drawings. The invention is capable of other embodiments and of being practiced and carried out in various ways. Also, it is understood that the phraseology and terminology employed herein are for the purpose of description and should not be regarded as limiting. As such, those skilled in the art will appreciate that the conception, upon which disclosure is based, may readily be utilized as a basis for designing other structures, methods, and systems for carrying out the several purposes of the present invention. It is important, therefore, that the claims be regarded as including such equivalent constructions insofar as they do not depart from the spirit and scope of the present invention.

### BRIEF DESCRIPTION OF THE DRAWINGS

The invention will be better understood when consideration is given to the following detailed description thereof. Such description makes reference to the annexed drawings wherein:

FIG. 1 is a perspective left-front view of an orthopedic chair for treatment and prevention of spinal disease according to the invention.

FIG. 2 is a perspective right-front view of the chair shown in FIG. 1

FIG. 3 is the same presentation as in FIG. 1 but with reference numbers.

FIG. 4 is the front view of the chair shown in FIG. 1

FIG. 5 is a left view of the chair shown in FIG. 1, with an occupant sitting on the chair.

FIG. 6 is a cross-section front view of the seat of the chair shown in FIG. 5 taken along lines A-A

FIG. 7 is a cross-section front view of the seat of the chair shown in FIG. 5 taken along lines B-B

FIG. 8 is a cross-section top view of the back seat of the chair shown in FIG. 5 taken along lines C-C

FIG. 9 is a cross-section top view of the back seat of the chair shown in FIG. 5 taken along lines D-D

FIG. 10 is a cross-section top view of the back seat of the chair shown in FIG. 5 taken along lines E-E

### DETAILED DESCRIPTION OF THE INVENTION

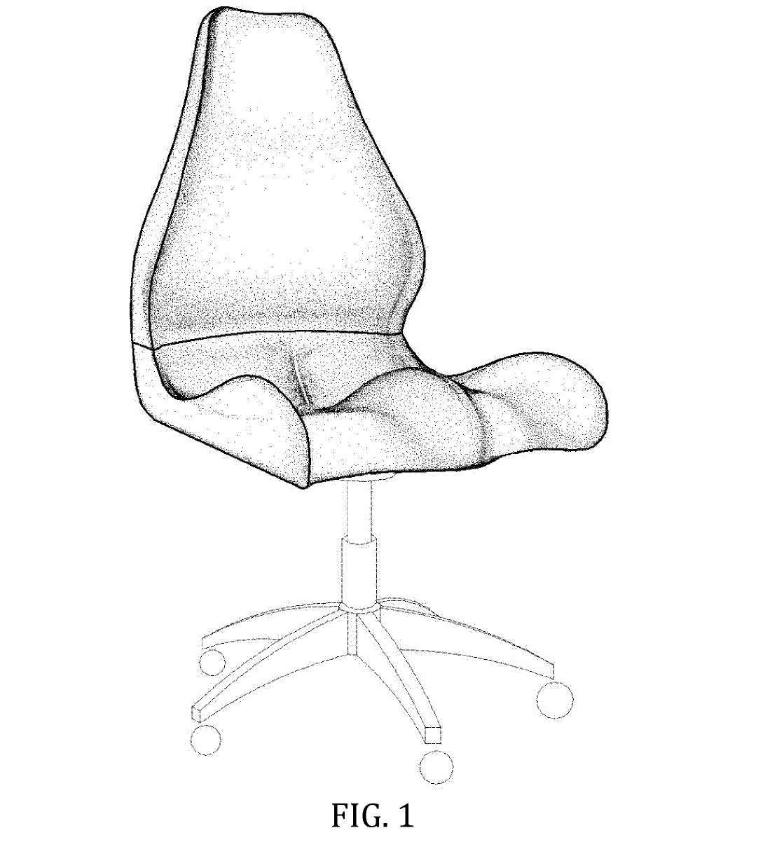

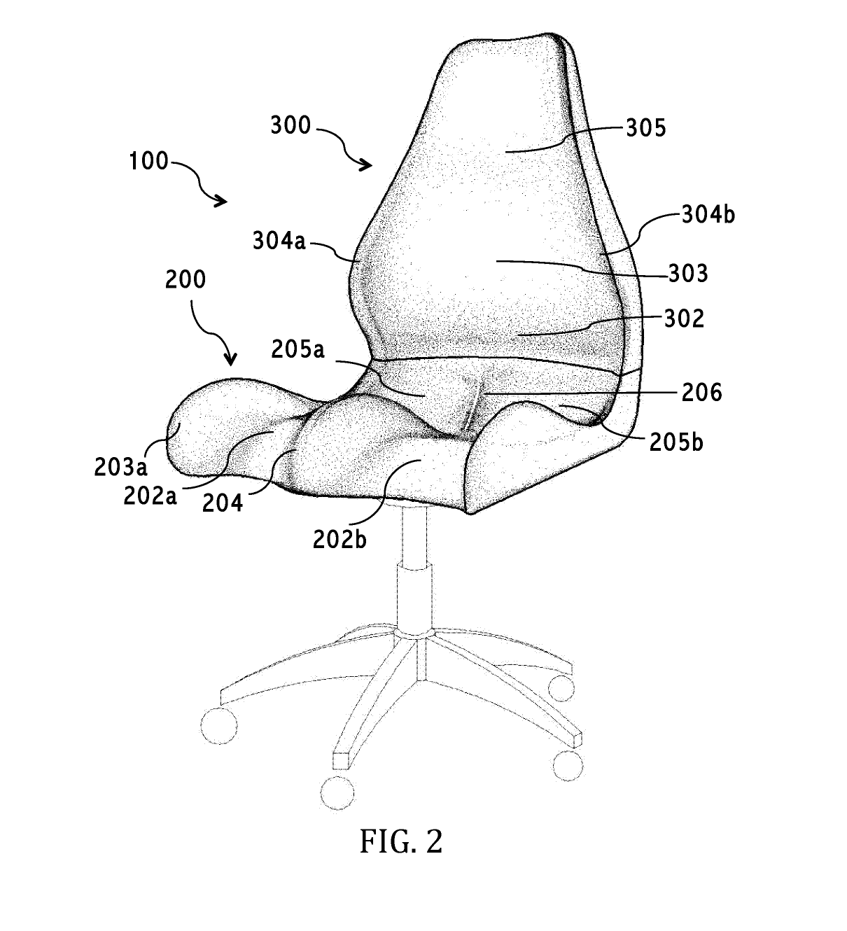

The present invention provides an orthopedic chair that is capable of treating and preventing spinal diseases. Referring to FIG. 1, the said chair 100 comprises a seat 200 and a seat back 300. The said seat 200 is mounted on a support that can include well-known height adjustment means; the support will not be described herein as it is not included in the invention.

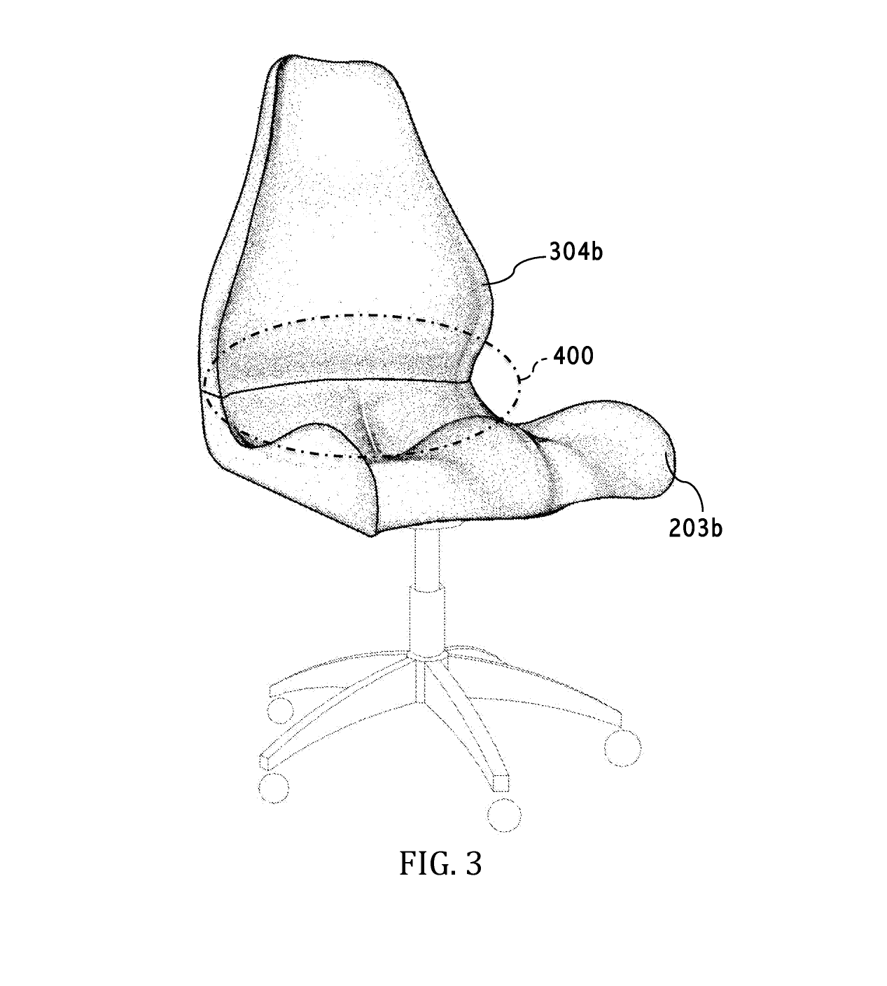

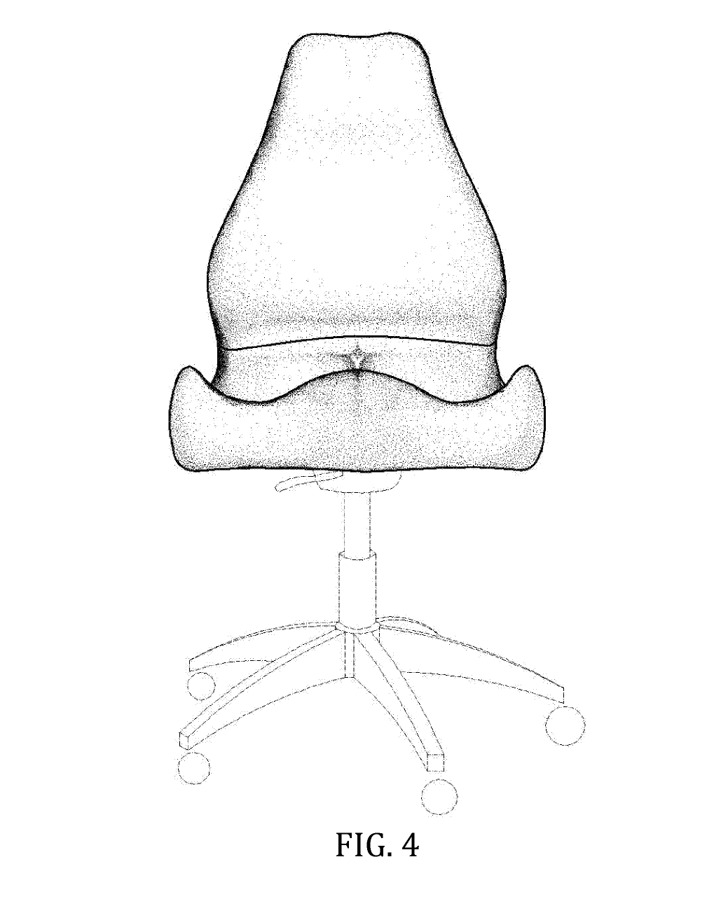

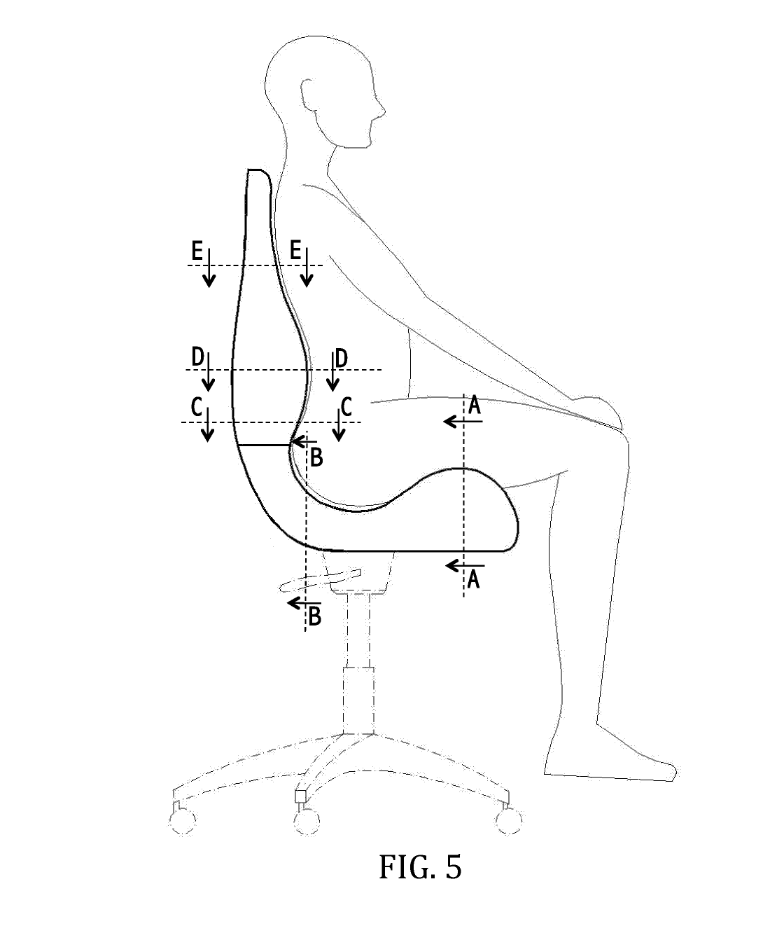

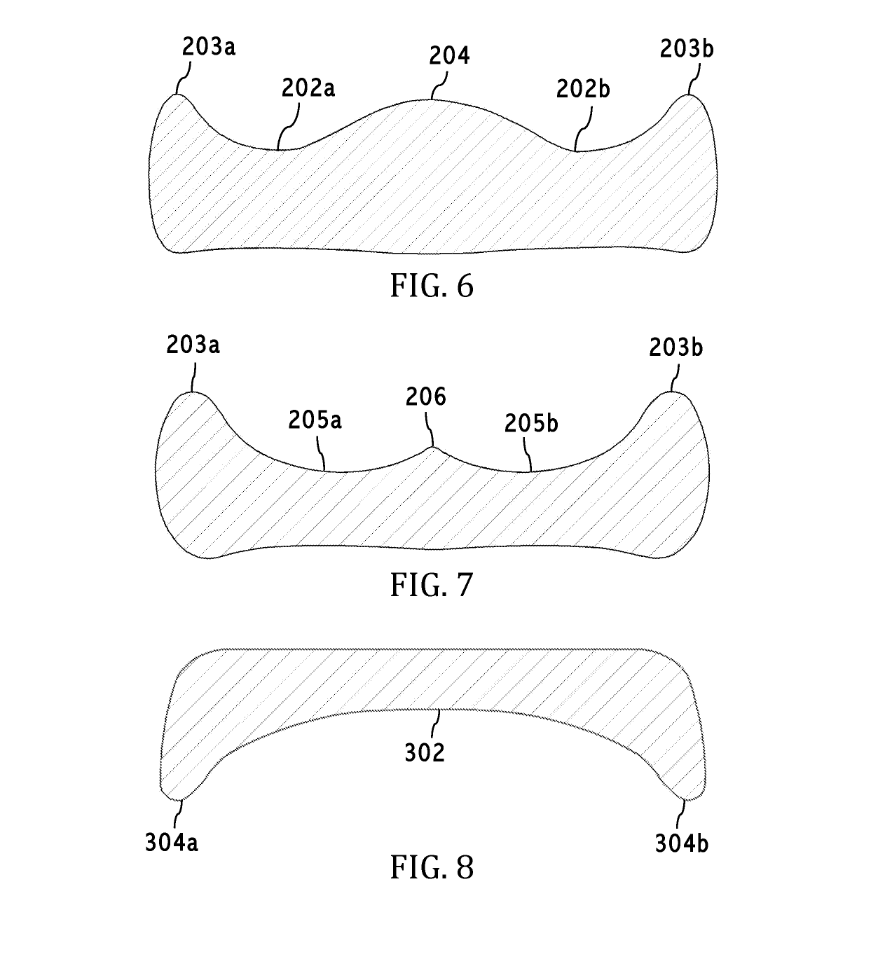

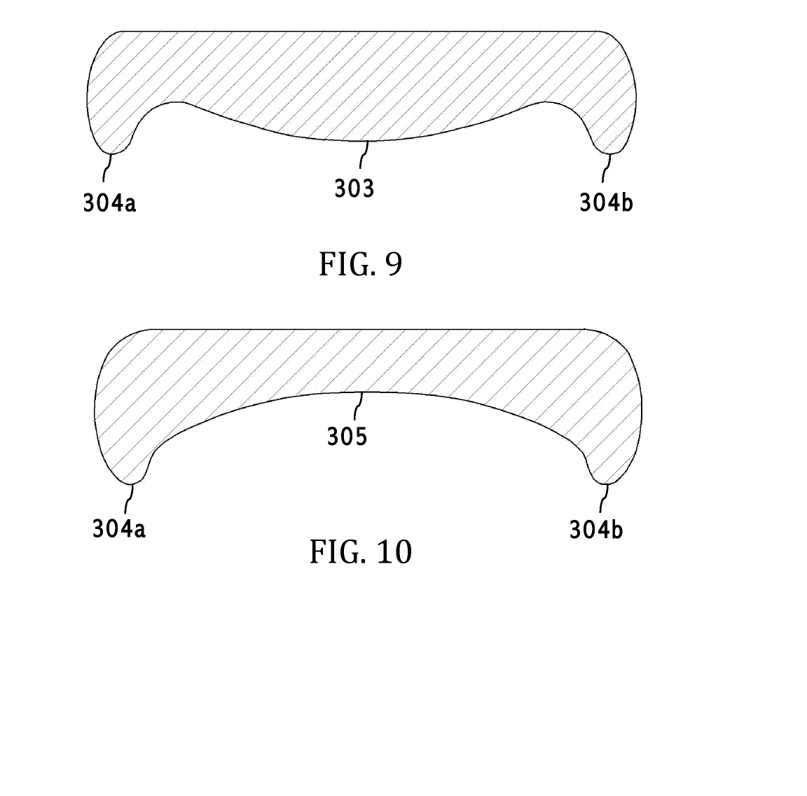

The said seat 200 has a shape that matches the natural contours of an occupant's buttocks, thighs, and upper legs. In which, the said seat has its concave backward portion, consisting of concave zone 205a and concave zone 205b, connect to the concave lowest portion 302 of the said seat back 300, creating a dedicated concave zone 400 as circled in FIG. 3. The dedicated concave zone 400 contours the occupant's buttocks closely but comfortably. In the center of the seat's concave backward portion, i.e. between concave zone 205a and concave zone 205b, is a small hump 206 that fits tightly into the occupant's sacrum and coccyx if his buttocks is fitting inside the dedicated concave zone 400.

The forward portion of the said seat 200 has a wide W-shape that contours the occupant's upper legs comfortably. In which, the two concave zones 202a and 202b of the seat's W-shaped forward portion contour the back of the occupant's upper legs. The center zone 204 of the seat's W-shaped forward portion, i.e. between the two concave zones 202a and 202b, is convex and has two functions. The first function is to snuggle the occupant's thighs comfortably. The second function is to encourage the occupant to fit his buttocks inside the dedicated concave zone 400, as sitting on the convex center zone 204 is not comfortable.

Furthermore, to ensure that the occupant always fit his buttocks inside the dedicated concave zone 400 while seated, the seat 200 is designed to be inclined backwardly in such a way that, when the occupant intentionally sits on the forward portion of the seat 200, his buttocks, pushed by the gravity force of his body, will gradually glide backward and fit themselves into the dedicated concave zone 400 eventually.

The left side 203a and the right side 203b of the W-shaped forward portion of the seat 200 are convex so that they contour the outer sides of the occupant's upper legs. Moreover, the left side 203a and the right side 203b encourage the occupant keeps his upper legs in a correct position, i.e. inside the concave zones 202a and 202b, since putting the upper legs on top of the two convex sides 203a and 203b is not comfortable. The said seat back 300 is designed in such a way that it contours the occupant's back tightly but not forcefully so that the occupant can sit in a correct posture comfortably. The seat back 300 comprises a lowest portion 302, as already described above, that is concave to contour the occupant's buttocks, a middle portion 303 that is convex to contour the lumbar vertebrae, and an upper portion 305 that is concave to contour the thoracic vertebrae. The left edge 304a and right edge 304b of the seat back 300 are convex so that the occupant's back cannot bend left or right therefore must stay straight up. The seat back 300 is height-adjustable to ensure that it well contours the occupant's spine.

Here, a proper sitting posture is defined as a posture in which the occupant sits on the seat 200 of the said chair 100 in such a way that his buttocks fit inside the dedicated concave zone 400, his sacrum and coccyx are contoured tightly by the small hump 206, his two upper legs fit inside the two concave zones 202a and 202b and is at an angle that is slightly greater than 90 degrees to the body's alignment, and his back stays at an angle close to verticality and is contoured tightly by the seat back 300. When an occupant is sitting in this proper sitting position, his pelvis is in neutral position, i.e. not rotating backward or forward, his spine is in its natural “S” shape and is not bent, and his sacrum and coccyx are not moved underneath.

Since the chair 100 keeps an occupant in a correct sitting posture, it is capable of preventing spinal diseases. Also, as the chair 100 is shaped to contour a non-diseased spine and is made of non-resilient materials, it is capable of treating spinal diseases including lumbar kyphosis, thoracic kyphosis, scoliosis, sacrum and coccyx misalignment, and herniated disc. For instance, if the occupant has lumbar kyphosis, the kyphotic lumbar vertebrae will apply a strong force onto the middle portion 303 of the seat back 300. This force is created by the body's gravity force that is re-directed backwardly as the buttocks and upper legs are glided backwardly due to the backwardly inclined seat. The middle portion 303, in return, applies a reaction force onto the kyphotic lumbar vertebrae. Overtime, the reaction force gradually pushes the kyphotic lumbar vertebrae back to its normal shape. Similarly, if the occupant's sacrum and coccyx are misaligned posteriorly, the small hump 206 will generate a reaction force greater than the one it generates in the case of naturally aligned sacrum and coccyx; therefore the small hump 206 will gradually push the misaligned sacrum and coccyx back to its natural position.

What is claimed is:

1. A chair comprises a seat and a seat back made of non-resilient materials, in which:
The seat has a shape that matches the natural contours of an occupant's buttocks, thighs, and upper legs, wherein: The seat's concave backward portion connects to the concave lowest portion of the said seat back, creating a dedicated concave zone that contours the occupant's buttocks closely; the seat's concave backward portion has in its center a small hump that contours tightly an occupant's sacrum and coccyx if his buttocks are fitting inside the dedicated concave zone; the seat's forward portion has a wide W-shape that contours the occupant's upper legs: the two concave zones of the seat's W-shaped forward portion contours the back sides of the occupant's upper legs, the very center zone of the seat's W-shaped forward portion is convex and snuggles the occupant's thighs; the seat is inclined backwardly in such a way that, when the occupant intentionally sits on the forward portion of the said seat, his buttocks, pushed by the gravity force of his body, will gradually glide backward and fit themselves into the said dedicated concave zone eventually; the left side and right side of the seat's W-shaped forward portion are convex so that they contour the outer sides of the occupant's upper legs.
The seat back contours the occupant's back, wherein: The seat back's lowest portion is concave to contour the occupant's buttocks; the middle portion that is convex to contour the lumbar vertebrae; the upper portion that is concave to contour the thoracic vertebrae; the left and right edges of the seat back are convex so that the occupant's back cannot bend left or right therefore must stay straight up; the seat back is height-adjustable to ensure that it well contours the occupant's spine; the seat back is positioned in a way that the occupant's back stays at an angle close to verticality, with his upper legs at an angle that is slightly greater than 90 degrees to the body's alignment.

2. The chair according to claim 1, in which the seat and seat back are made of low-resilient, rather non-resilient, materials to make the chair more comfortable to sit on.
